(19) United States
(12) Patent Application Publication
(10) Pub. No.: US 2024/0382252 A1
(43) Pub. Date: Nov. 21, 2024

(54) ANCHOR POINT FOR UMBILICAL OF SURGICAL ABLATION PROBE

(71) Applicant: Medtronic, Inc., Minneapolis, MN (US)

(72) Inventors: Hannah Jean Walter, Roseville, MN (US); Philip Soviak, Menomonie, WI (US)

(21) Appl. No.: 18/664,583

(22) Filed: May 15, 2024

Related U.S. Application Data
(60) Provisional application No. 63/502,507, filed on May 16, 2023.

(51) Int. Cl. A61B 18/14; A61B 18/00; A61B 90/00

(52) U.S. Cl. CPC A61B 18/1492; A61B 90/08; A61B 2018/00273

(57) ABSTRACT

An anchor point for an umbilical of a surgical ablation probe includes a main body, a first tab coupled to the main body and configured to flex relative to the main body about a first axis, a second tab coupled to the main body and configured to flex relative to the main body about a second axis, a third tab coupled to the first tab and configured to flex relative to the first tab about a third axis, and a fourth tab coupled the second tab and configured to flex relative to the second tab about a fourth axis. The first tab defines a third tab opening configured to be exposed when the third tab flexes relative to the first tab, and the second tab defines a fourth tab opening configured to be exposed when the fourth tab flexes relative to the second tab.

### CROSS-REFERENCE TO RELATED APPLICATIONS

This application claims priority to U.S. Application No. 63/502,507, filed May 16, 2023, the entire contents of which are incorporated herein by reference.

### FIELD

The present technology is generally related to surgical ablation probes, and to the umbilical of a surgical ablation probe.

### BACKGROUND

Surgical ablation probes commonly include an umbilical (e.g., elongated cord) that extends between a device console and a probe device. The probe device and the umbilical typically come packaged together, and are removed from the packaging prior to use.

### SUMMARY

The techniques of this disclosure generally relate to the use of an anchor point to control, position, and/or secure an umbilical of a surgical ablation probe. The anchor points are used to suspend the umbilical above the ground and away from medical personnel or other equipment in a surgical environment. The anchor points are additionally, or alternatively, used to secure the umbilical within the packaging and facilitate proper aseptic removal of the umbilical and its associated probe from the packaging.

In one aspect, the disclosure provides an anchor point for an umbilical of a surgical ablation probe, the anchor point including a main body, a first tab coupled to the main body and configured to flex relative to the main body about a first axis, a second tab coupled to the main body and configured to flex relative to the main body about a second axis, a third tab coupled to the first tab and configured to flex relative to the first tab about a third axis, and a fourth tab coupled the second tab and configured to flex relative to the second tab about a fourth axis. The first tab defines a third tab opening configured to be exposed when the third tab flexes relative to the first tab, and the second tab defines a fourth tab opening configured to be exposed when the fourth tab flexes relative to the second tab.

In another aspect, the disclosure provides an anchor point for an umbilical of a surgical ablation probe, the anchor point including a main body having a first attachment portion and a second attachment portion coupled to the first attachment portion. The first attachment portion includes a loop configured to receive a portion of the umbilical. The second attachment portion includes at least one of an aperture that extends through the main body or a layer of adhesive on the main body.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1 is a schematic illustration of a surgical ablation probe having a device console, a probe device, an umbilical extending between the device console and the probe device, and an anchor point according to one example coupled to the umbilical.

FIG. 2 is a perspective view of the anchor point coupled to the umbilical of the surgical ablation probe.

FIG. 3 is a front view of the anchor point.

FIG. 4 is a front view of three anchor points coupled to one another.

FIG. 5 is a schematic illustration of the umbilical, probe, and two anchor points within a packaging.

FIG. 6 is a perspective view of three anchor points according to another example, coupled to one another with perforations.

FIG. 7 is a front view of an anchor point according to another example.

FIG. 8 is a side view of the anchor point of FIG. 7, illustrating a plurality of tabs that are flexed relative to a main body of the anchor point.

FIG. 9 is a front view of the anchor point of FIG. 7, illustrating some of the openings formed by the tabs.

FIG. 10 is a perspective view of the anchor point of FIG. 7, along with the umbilical.

FIG. 11 is a perspective view of the anchor point from FIG. 7, partially coupled to the umbilical.

FIG. 12 is a perspective view of a set of three of the anchor points from FIG. 7, one of the anchor points being fully coupled to the umbilical.

FIG. 13 is a perspective view of the set of three anchor points from FIG. 9, each of the anchor points being fully coupled to the umbilical.

### DETAILED DESCRIPTION

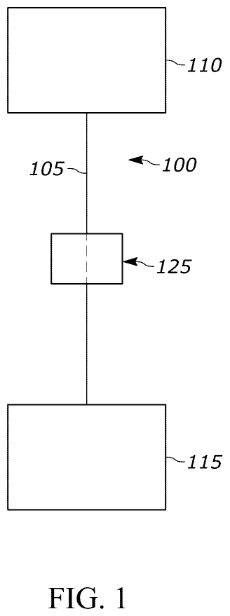

With reference to FIGS. 1-4, a medical device, illustrated as a surgical ablation probe 100, includes an umbilical 105 and a probe device 110 coupled (e.g., releasably coupled) to the umbilical 105. In the illustrated example, the surgical ablation probe 100 also includes a device console 115. The umbilical 105 extends between and is coupled (e.g., releasably coupled) to both the probe device 110 and the device console 115. In some examples, the umbilical 105 and the probe device 110 come packaged together (e.g., in a sterilized packaging), and are removed from the packaging prior to use.

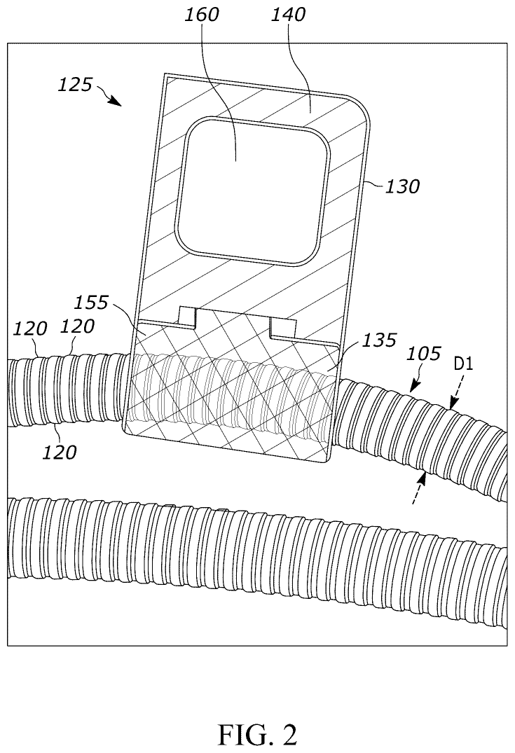

The umbilical 105 is any type of cord or cable or other structure (e.g., flexible, coaxial cable) that is used during a surgical event for example to deliver or remove material (e.g., fluid or gas) from a surgical site on the body, or generally control movement of the probe device 110, or otherwise assist a surgeon or other medical personnel during the surgical event. In the illustrated example, the umbilical 105 is an elongate, flexible cable, having ribs 120 (e.g., separated annular ribs 120 as seen in FIG. 2) to provide flexibility. The umbilical 105 is coupled to the device console 115, and the device console 115 includes for example a source of cryogenic material (e.g., liquid refrigerant) that is passed through the umbilical 105 to the probe device 110.

The surgical ablation probe 100 may be any type of surgical ablation probe, including for example a cryoablation probe. In some examples, the surgical ablation probe 100 is used to ablate tissue, and the probe device 110 includes for example a radiofrequency (RF) electrode used for ablation, or a probe tip (e.g., malleable tip) that is cooled with cryoablation gas (e.g., Argon) for ablation.

With continued reference to FIGS. 1-4, the surgical ablation probe 100 also includes at least one anchor point 125. As described further herein, the anchor points 125 are used to control, position, and/or secure the umbilical 105. For example, the anchor points 125 may be used to suspend the umbilical 105 above the ground and away from medical personnel or other equipment in a surgical environment. The anchor points 125 may additionally, or alternatively, be used to secure the umbilical 105 in a packaging and facilitate removal of the umbilical 105 and its associated probe device 110 from the packaging.

With reference to FIGS. 2-4, in the illustrated example each anchor point 125 includes a main body 130. In the illustrated example, the main body 130 has a generally rectangular outer profile (with the exception of a lower hook 145 described below) when the main body 130 is spread out flat, although other examples include other shapes than that illustrated. In the illustrated example, the main body 130 is thin, and has an overall width W and an overall height H that are each larger than a thickness of the main body 130. The height H is larger than the width W, such that a ratio of the height H to the width W is at least 2:1, at least 3:1, between 2:1 and 5:1, or other values and ranges. Other examples include other shapes and sizes. The anchor point 125, including the main body 130, may be formed for example from a flexible material (e.g., a thin plastic material, such as high density polyethylene (HDPE) or other suitable material). In some examples, one or more of the anchor points 125 are sterilized prior to use.

With continued reference to FIGS. 2-4, the main body 130 includes a primary attachment portion 135 for coupling the anchor point 125 to the umbilical 105, and a secondary attachment portion 140 for coupling the anchor point 125 to a wall, curtain, or other structure within a surgical environment. The primary attachment portion 135 is coupled (e.g., directly coupled) to the secondary attachment portion 140. In the illustrated example, the primary attachment portion 135 is integrally formed as a single piece with the secondary attachment portion 140.

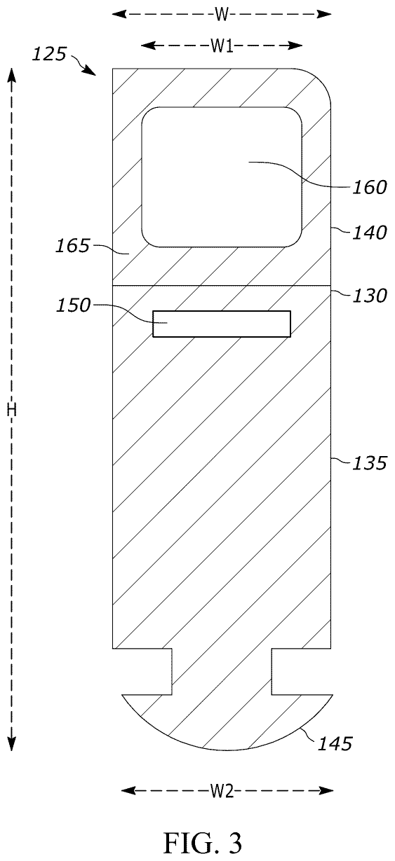

With continued reference to FIGS. 2-4, in the illustrated example the primary attachment portion 135 includes a lower hook 145, and a slot 150. The slot 150 is sized and shaped to receive the lower hook 145, and the lower hook 145 is sized and shaped, and made for example of a flexible material (e.g., a thin plastic), to slide through the slot 150 and form a loop 155 (FIG. 2). In the illustrated example, the slot has a first width W1 (FIG. 3), and the lower hook 145 has a second width W2 that is larger than the first width W1, such that the flexible lower hook 145 is first flexed (e.g., bent) to pass through the slot 150, and then returns to its natural shape and is secured behind the slot 150. As seen in FIG. 2, the loop 155 is large enough to wrap around and/or receive a portion of the umbilical 105. In some examples, the umbilical 105 has a diameter D1, and the loop 155 forms an opening that is larger than the diameter D1 (e.g., less than 50% larger, less than 40% larger, less than 30% larger), to securely hold the umbilical 105 and inhibit significant movement of the umbilical 105 relative to the anchor point 125. In some examples, the umbilical 105 extends (e.g., wraps), multiple times through the same loop 155. Accordingly, the loop 155 may form an opening that is at least twice as large as the diameter D1. In some examples, the lower hook 145 and the slot 150 are removed, and the primary attachment portion 135 includes a loop 155 that is formed integrally as a single piece with the rest of the of the anchor point 125, or includes a loop 155 that is formed in ways other than the use of a lower hook 145 and slot 150.

With reference to FIG. 3, in the illustrated example the secondary attachment portion 140 is positioned at an opposite end of the main body 130 from the lower hook 145 (e.g., when the anchor point 125 is positioned flat and the loop 155 has not yet been formed). The secondary attachment portion 140 includes an aperture 160 that is sized and shaped for example to fit over a corresponding structure within the surgical environment (e.g., a hook, post), to suspend the anchor point 125 (and the attached umbilical 105) within the surgical environment above the ground and away for example from medical personnel or other equipment. In some examples, a portion of a surgical curtain may be passed through the aperture 160, to suspend the anchor point 125 in place within the surgical environment. Other examples include other numbers, locations, and sizes of apertures 160 than that illustrated. In some examples, the secondary attachment portion 140 includes multiple apertures 160.

With reference to FIGS. 2-4, in some examples the secondary attachment portion 140 additionally, or alternatively, includes at least one layer of adhesive 165 (FIG. 3) on the main body 130, for example on a front and/or back side of the secondary attachment portion 140. In the illustrated example, the layer of adhesive 165 extends entirely around the aperture 160, although in other examples the layer of adhesive 165 extends only partially around the aperture 160. The layer(s) of adhesive 165 may be used, for example, to couple the anchor point 125 to the wall, curtain, or other structure within a surgical environment, and to suspend at least a portion of the umbilical 105 in place above the ground and away for example from medical personnel or other equipment. In some examples, the secondary attachment portion 140 does not include any apertures 160. Rather, the secondary attachment portion 140 only includes one or more layers of the adhesive 165. In other examples, the secondary attachment portion 140 includes one or more apertures 160, but no layers of adhesive 165. In yet other examples, the secondary attachment portion 140 includes at least one aperture 160, and also at least one layer of adhesive 165.

In yet other examples, the anchor point 125 does not include a secondary attachment portion 140 for attaching the anchor point 125 to a wall, curtain, or other structure within a surgical environment. Rather, the anchor point 125 only includes the primary attachment portion 135, and is only used for example to receive and/or hold together portions of the umbilical 105 (e.g., in a packaging prior to use, and/or during use).

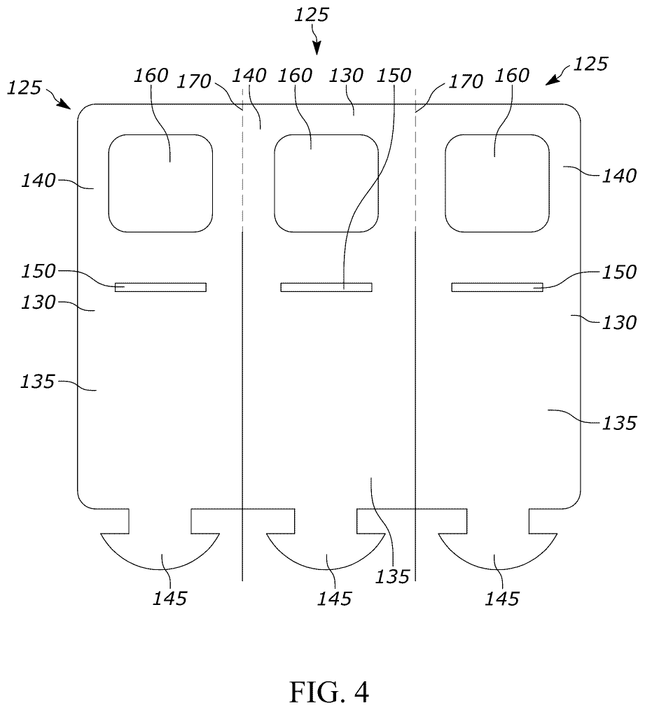

With reference to FIG. 4, in some examples multiple anchor points 125 are initially coupled to one another (e.g., during a manufacturing process), and perforations 170 are provided to tear the anchor points 125 apart from one another for individual use. The illustrated example shows three anchor points 125 initially coupled together, but other examples include different numbers (e.g., two anchor points 125, four anchor points 125, five anchor points 125, etc.).

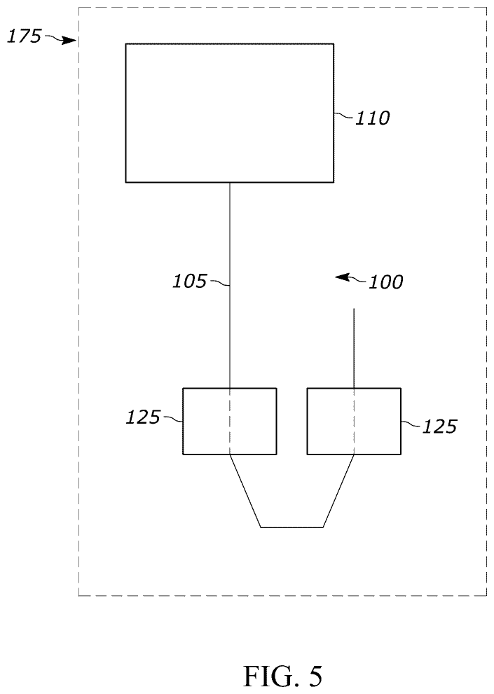

With reference to FIG. 5, and as described above, in some examples, one or more anchor points 125 are coupled to the umbilical 105 within a packaging 175. For example, and as described above, the umbilical 105 and the probe device 110 may initially be packaged together. The umbilical 105 may extend (one or more times) through one or more of the loops 155 and one or more of the anchor points 125 described above, to hold the umbilical 105 in place within the packaging 175. When the packaging 175 is opened, the umbilical 105 and the probe device 110 may be removed, with the anchor points 125 still securely holding onto the umbilical 105. The anchor points 125 are then secured to points within the surgical environment (e.g., using the aperture 160 and/or layers of adhesive 165) to suspend the umbilical 105 and keep it away for example from the ground and from medical personnel.

In some examples, two or more anchor points 125 remain coupled together within the packaging 175. Once the packaging 175 is opened, the umbilical 105 and the probe device 110 are then removed, and the anchor points 125 are then separated from one another (e.g., using the perforations 170), and secured to different points within the surgical environment.

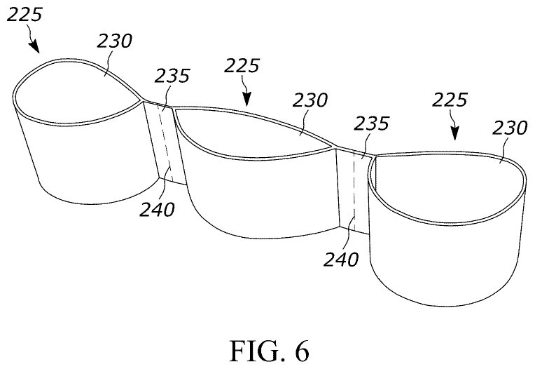

FIG. 6 illustrates another set of anchor points 225. The anchor points 225 each include loops 230 (e.g., forming at least part of a primary attachment portion), similar to the loops 155 described above. The loops 230 form openings that are sized and shaped to receive the umbilical 105 (e.g., one or more windings of the umbilical 105). As illustrated in FIG. 6, each of the anchor points 225 is separated by a flange 235 (e.g., forming at least part of a secondary attachment portion), and by perforations 240 that extend along each flange 235. Similar to the perforations 170 described above, the perforations 240 may be used to separate the anchor points 225. For example, the set of anchor points 225 seen in FIG. 5 may initially remain together within the packaging 175. The umbilical 105 passes one or more times through one or more (or each) of the loops 230. Once the packaging 175 is opened, the umbilical 105 (and the probe device 110) are then removed from the packaging 175. The anchor points 225 are then torn apart from one another, using the perforations 240, and the anchor points 225 may then be secured to different locations within the surgical environment (e.g., using layers of adhesive that are on the flanges 235 or other portions of the anchor points 225). Similar to the anchor points 125, the anchor points 225 may be formed for example from a flexible material (e.g., a thin plastic material, such as HDPE or other suitable material). In some examples, one or more of the anchor points 225 are sterilized prior to use.

In some examples, the anchor points 225 are not attached to a wall, curtain, or other structure within a surgical environment, and do not include adhesive on the flanges 235 (or apertures such as apertures 160). Rather, the anchor points 225 are only used for example to receive and/or hold together portions of the umbilical 105 (e.g., in a packaging prior to use, and/or during use).

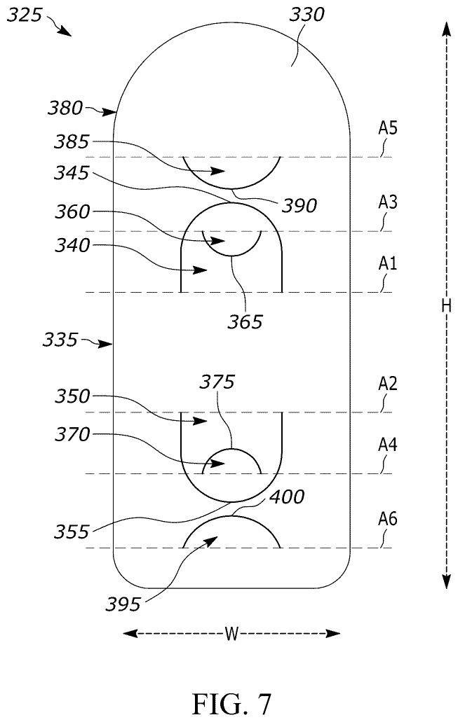

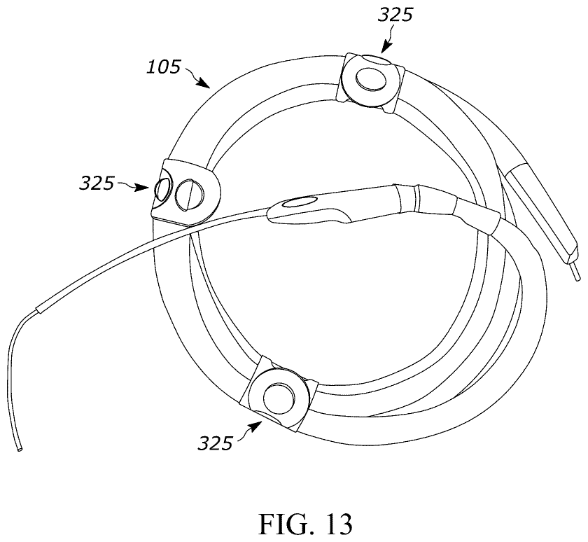

FIGS. 7-13 illustrate another example of a set of anchor points 325. Similar to the anchor points 125 and 225 described above, the anchor points 325 may also be used to control, position, and/or secure the umbilical 105. For example, the anchor points 325 may be used to suspend the umbilical 105 above the ground and away from medical personnel or other equipment in a surgical environment. The anchor points 325 may additionally, or alternatively, be used to secure the umbilical 105 in a packaging and facilitate removal of the umbilical 105 and its associated probe device 110 from the packaging.

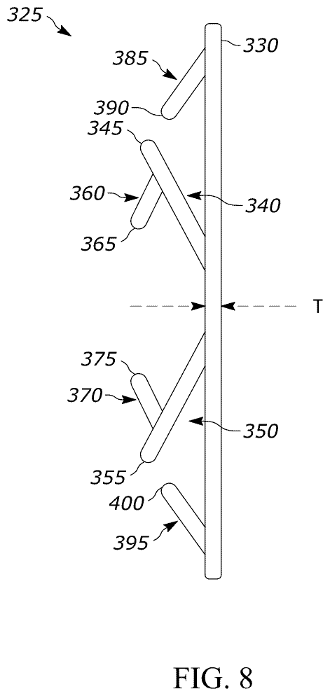

With reference to FIGS. 7 and 8, each anchor point 325 includes a main body 330. In the illustrated example, the main body 330 has a generally rectangular outer profile (with the exception of a rounded upper end), and has an overall width W and an overall height H (FIG. 7) that are each larger than a thickness T (FIG. 8) of the main body 330. The height H is larger than the width W, such that a ratio of the height H to the width W is at least 2:1, at least 3:1, between 2:1 and 5:1, or other values and ranges. Other examples include different shapes and sizes for the main body 330 than that illustrated. The anchor point 325, including the main body 330, may be formed for example from a flexible material (e.g., a thin plastic material, such as HDPE or other suitable material). In some examples, one or more of the anchor points 325 are sterilized prior to use.

With reference to FIGS. 7-13, the anchor point 325 may include one or more primary attachment portions for coupling the anchor point 325 to the umbilical 105, and/or one or more secondary attachment portions for coupling the anchor point 325 to a wall, curtain, or other structure within a surgical environment.

For example, and with reference to FIG. 7, the anchor point 325 may include a first primary attachment portion 335 (e.g., a central portion) for coupling the anchor point 325 to a first region of the umbilical 105 (e.g., to a first winding or windings of the umbilical 105). In the illustrated example, the first primary attachment portion 335 includes a first tab 340 that is flexible relative to the main body 330. As illustrated in FIGS. 7 and 8, the first tab 340 is flexible about a first axis A1, the first axis A1 extending along an interface where the first tab 340 connects to the main body 330. The first tab 340 includes a first free end 345 spaced opposite the first axis A1. The first tab 340 may be a cut-out region of the main body 330. In the illustrated example, the first tab 340 has generally a U-shape, although other examples may include other shapes.

With continued reference to FIGS. 7 and 8, the first primary attachment portion 335 also includes a second tab 350 that is flexible relative to the main body 330. The second tab 350 is flexible about a second axis A2, the second axis A2 extending along an interface where the second tab 350 connects to the main body 330. As illustrated in FIGS. 7 and 8, the second tab 350 includes a second free end 355 spaced opposite the second axis A2. The first axis A1 and the second axis A2 are each positioned between the first free end 345 and the second free end 355. The second tab 350 may be a cut-out region of the main body 330. In the illustrated example, the second tab 350 has generally a U-shape, although other examples include other shapes.

With continued reference to FIGS. 7 and 8, the first primary attachment portion 335 also includes a third tab 360 that is flexible relative to the first tab 340. The third tab 360 is flexible about a third axis A3, the third axis A3 extending along an interface where the third tab 360 connects to the first tab 340. As illustrated in FIGS. 7 and 8, the third tab 360 includes a third free end 365 spaced opposite the third axis A3. The third tab 360 may be a cut-out region of the first tab 340. In the illustrated example, the third tab 360 has generally a U-shape, although other examples include other shapes.

With continued reference to FIGS. 7 and 8, the first primary attachment portion 335 also includes a fourth tab 370 that is flexible relative to the second tab 350. The fourth tab 370 is flexible about a fourth axis A4, the fourth axis A4 extending along an interface where the fourth tab 370 connects to the second tab 350. As illustrated in FIGS. 7 and 8, the fourth tab 370 includes a fourth free end 375 spaced opposite the fourth axis A4. The fourth free end 375 faces the third free end 365. The first axis A1 and the second axis A2 are each positioned between the third free end 365 and the fourth free end 375. The fourth tab 370 may be a cut-out region of the second tab 350. In the illustrated example, the fourth tab 370 has generally a U-shape, although other examples include other shapes.

With continued reference to FIGS. 7 and 8, the anchor point 325 may also include a second primary attachment portion 380 (e.g., outer portion) for coupling the anchor point 325 to a second region of the umbilical (e.g., to a second winding or windings of the umbilical). In the illustrated example, the second primary attachment portion 380 includes a fifth tab 385 that is flexible relative to the main body 330. The fifth tab 385 is flexible about a fifth axis A5, the fifth axis A5 extending along an interface where the fifth tab 385 connects to the main body 330. As illustrated in FIGS. 7 and 8, the fifth tab 385 includes a fifth free end 390 spaced opposite the fifth axis A5. The fifth free end 390 faces the first free end 345. The fifth tab 385 may be a cut-out region of the main body 330. In the illustrated example, the fifth tab 385 has a generally “U” shape, although other examples may include other shapes.

With continued reference to FIGS. 7 and 8, the second primary attachment portion 380 also includes a sixth tab 395 that is flexible relative to the main body 330. The sixth tab 395 is flexible about a sixth axis A6, the sixth axis A6 extending along an interface where the sixth tab 395 connects to the main body 330. In the illustrated example each of the first axis A1, the second axis A2, the third axis A3, the fourth axis A4, the fifth axis A5, and the sixth axis A6 are parallel to one another, although in other examples one or more the first axis A1, the second axis A2, the third axis A3, the fourth axis A4, the fifth axis A5, or the sixth axis A6 maybe arranged differently (e.g., may be skewed relative another of the axes). As illustrated in FIGS. 7 and 8, the sixth tab 395 includes a sixth free end 400 spaced opposite the sixth axis A6. The first tab 340, the second tab 350, the third tab 360, and the fourth tab 370 are each positioned between the fifth free end 390 and the sixth free end 400. The sixth free end 400 faces the second free end 355. The sixth tab 395 may be a cut-out region of the main body 330. In the illustrated example, the sixth tab 395 has a generally “U” shape, although other examples may include other shapes.

With reference to FIG. 8, and as described above, each of the first tab 340, the second tab 350, the third tab 360, the fourth tab 370, the fifth tab 385, and the sixth tab 395 is flexible relative to the main body 330, and may flex/pivot outwardly away from the main body 330 in one or more directions (e.g., in forward direction as shown in FIG. 8 and/or in a backward direction).

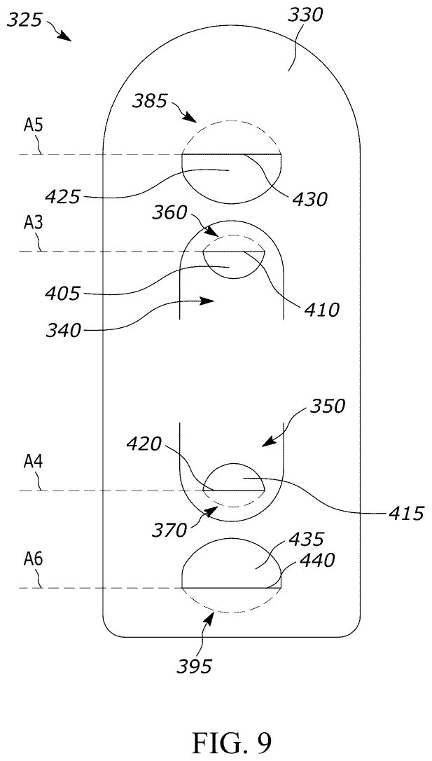

With reference to FIG. 9, in some examples the third tab 360 may flex about the third axis A3, leaving behind and exposing a third tab opening 405 defined by the first tab 340. The third tab opening 405 is bounded, in part, by a third tab edge 410 (e.g., a straight edge). In the illustrated example, the third axis A3 extends along this third tab edge 410.

With continued reference to FIG. 9, the fourth tab 370 may flex about the fourth axis A4, leaving behind and exposing a fourth tab opening 415 defined by the second tab 350. The fourth tab opening 415 is bounded, in part, by a fourth tab edge 420 (e.g., a straight edge). In the illustrated example, the fourth axis A4 extends along this fourth tab edge 420.

With continued reference to FIG. 9, the fifth tab 385 may flex about the fifth axis A5, leaving behind and exposing a fifth tab opening 425 defined the main body 330. The fifth tab opening 425 is bounded, in part, by a fifth tab edge 430 (e.g., a straight edge). In the illustrated example, the fifth axis A5 extends along this fifth tab edge 430.

With continued reference to FIG. 9, the sixth tab 395 may flex about the sixth axis A6, leaving behind and exposing a sixth tab opening 435 in the main body 330. The sixth tab opening 435 is bounded, in part, by a sixth tab edge 440 (e.g., a straight edge). In the illustrated example, the sixth axis A6 extends along this sixth tab edge 440.

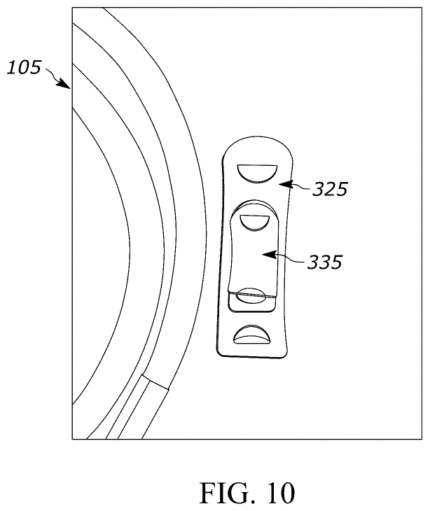

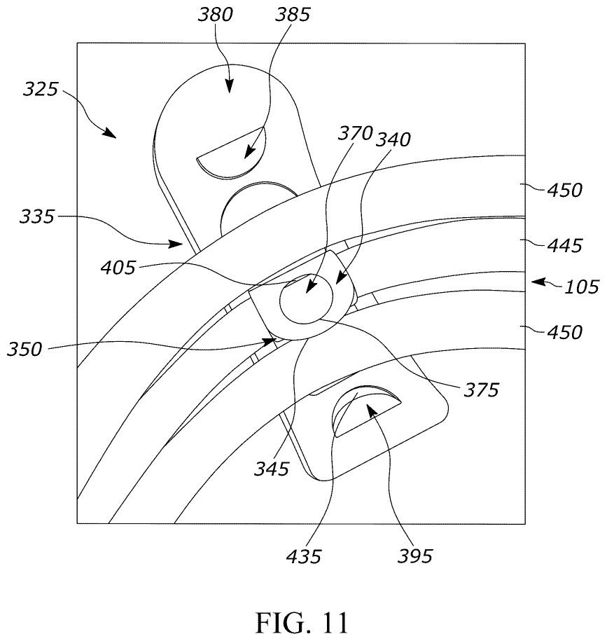

With reference to FIGS. 10 and 11, and as described above, the first primary attachment portion 335 may couple to a first portion 445 of the umbilical 105 (e.g., a first winding). For example, the first tab 340 and the second tab 350 may be flexed about the first axis A1 and the second axis A2, respectively, and wrapped about the first portion 445 of the umbilical 105 (e.g., forming a loop), such that the first free end 345 of the first tab 340 and the second free end 355 of the second tab 350 overlap (FIG. 11).

With reference to FIGS. 9 and 11, in this position, the third tab 360 may be passed through the fourth tab opening 415, and the fourth tab 370 may be passed through the third tab opening 405, in opposite directions (e.g., such that the third tab edge 410 is aligned with the fourth tab edge 420). In this position, the first primary attachment portion 335 is interlocked, the first and second tabs 340, 350 are inhibited from being pulled apart, and the first portion of the umbilical 105 is secured.

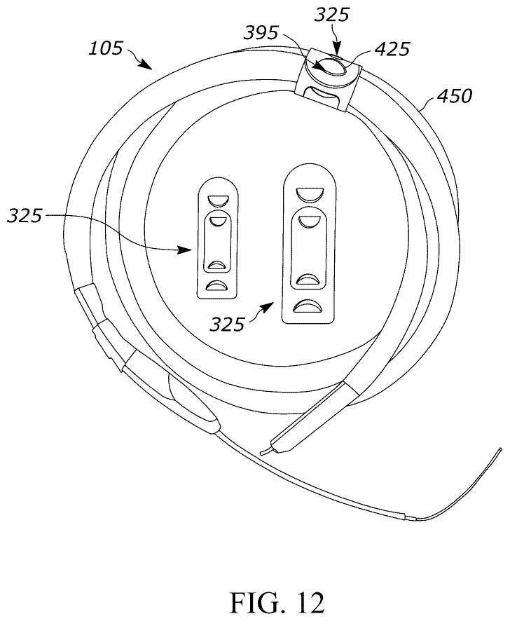

With reference to FIGS. 11 and 12, and as described above, the second primary attachment portion 380 may couple to a second portion 450 (or portions) of the umbilical 105 (e.g., a second and/or third winding). For example, the overall main body 330 may wrap about the second portion or portions 450 of the umbilical 105 (e.g., forming a loop), such that the main body 330 also extends around the first portion 445 of the umbilical 105.

With reference to FIGS. 9, 11, and 12, in this position, the fifth tab 385 may be passed through the sixth tab opening 435, and the sixth tab 395 may be passed through the fifth tab opening 425, in opposite directions (e.g., such that the fifth tab edge 430 is aligned with the sixth tab edge 440). In this position, the second primary attachment portion 380 is interlocked, the main body 330 is inhibited from being pulled apart, and the second portion (or portions 450) of the umbilical 105 is secured.

In some examples, the anchor points 325 may be used to initially package the umbilical 105. Use of one or more of the anchor points 325 (e.g., three anchor points 325) in a packaging (e.g., the packaging 175) may help to tightly hold the umbilical 105 in a coiled configuration, for ease of packaging. Once the umbilical 105 is removed from the packaging, the anchor points 325 may then also be used to anchor the umbilical 105 and/or suspend the umbilical within a surgical environment. For example, the fifth tabs 385 may be passed back through and out of the sixth tab openings 435, and the sixth tabs 395 may be passed back through and out of the fifth tab openings 425, in opposite directions, and the main bodies 330 may be unwrapped from the second portion or portions 450 of the umbilical 105 once the umbilical 105 has been removed from a package. The fifth tab openings 425 and/or the sixth tab openings 435 may then be used to suspend and hold the umbilical 105. For example, one or more curtains, clamps, or other structure may be passed through the fifth tab openings 425 and/or the sixth tab openings 435. In some examples, a clamp or other structure may additionally or alternatively physically grasp ends of the unwrapped main body 330, to suspend and hold the umbilical 105.

The features of each of the anchor points 125, 225, 325 described herein may be used in combination, or with other anchor points. For example, one or more of the anchor points 225 from FIG. 6 may include an attachment portion similar to the secondary attachment portion 140 in FIGS. 2-4, with an aperture and/or layer of adhesive to facilitate coupling the anchor point 225 to a wall, curtain, or other structure within a surgical environment. Similarly, one or more of the anchor points 325 from FIGS. 7-13 may include an attachment portion similar to the secondary attachment portion 140 in FIGS. 2-4, with an aperture and/or layer of adhesive to facilitate coupling the anchor point 325 to a wall, curtain, or other structure within a surgical environment. Additionally, two or more of the anchor points 325 from FIGS. 7-13 may initially be coupled together with perforations, similar to the perforations 170 in FIG. 4, or the perforations 240 in FIG. 6. Additionally, the anchor points 125 and/or the anchor points 225 may include multiple primary attachment portions, similar to the first and second primary attachment portions 335, 380 seen in the anchor point 325 of FIGS. 7-13, for coupling the anchor points 125, 225 to different regions of the umbilical 105.

While FIG. 5 illustrates the anchor points 125 within the overall packaging 175, any of the anchor points 125, 225, 325 described herein may be used within the packaging 175. In some examples, some of the anchor points 125, 225, 325 may be different from one another within the packaging 175, or outside of the packaging 175. For example, one or more of the anchor points within the packaging 175 may be an anchor point such as the anchor point 325 from FIGS. 7-13, while one or more of the remaining anchor points within the packaging 175 may be the anchor point 125 from FIGS. 2-4, or the anchor point 225 from FIG. 6. Accordingly, the packaging 175 may incorporate the use of a variety of different anchor points. Similarly, during a surgical procedure, a variety of different anchor points 125, 225, 325 may also, or alternatively, be used to secure and/or hold the umbilical 105.

Additionally, any of the anchor points 125, 225, 325 described herein may be used during a manufacturing process or in a final packaged assembly to maintain and/or control a length of the umbilical 105. For example, and as described above, the anchor points 125, 225, 325 may include one or more regions (e.g., loops) through which one or more portions of the umbilical 105 extend, and multiple anchor points 125, 225, 325 may be coupled to the umbilical 105. In some examples, the anchor points 125, 225, 325 engage the umbilical 105 so as to hold the umbilical 105 in a coiled configuration (e.g., while inside of the packaging 175). An umbilical having a length of 10 feet, for example, may be reduced to coiled shape having a diameter or width of 8 inches. Keeping the umbilical 105 in a coiled configuration may help to maintain a product workflow, and/or maintain production space, and/or reduce space required within the packaging 175.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

The examples may be further described by reference to the following numbered paragraphs:

1. An anchor point for an umbilical of a surgical ablation probe, the anchor point comprising:
a main body having a first attachment portion and a second attachment portion coupled to the first attachment portion;
wherein the first attachment portion includes a loop configured to receive a portion of the umbilical;
wherein the second attachment portion includes at least one of an aperture that extends through the main body or a layer of adhesive on the main body.

2. The anchor point of paragraph 1, wherein the first attachment portion includes a lower hook and a slot, wherein the lower hook is configured to be inserted through the slot to form the loop.

3. The anchor point of paragraph 2, wherein the slot has a first width and the lower hook has a second width greater than the first width, such that the lower hook is configured to be flexed to pass through the slot, and is configured to then return to a natural shape and be secured behind the slot to form the loop.

4. The anchor point of paragraph 1, wherein the second attachment portion includes the aperture, wherein the aperture is sized and shaped to fit over a corresponding structure within a surgical environment.

5. The anchor point of paragraph 1, wherein the second attachment portion includes the layer of adhesive, wherein the layer of adhesive is configured to couple the anchor point to a wall, curtain, or other structure within a surgical environment.

6. The anchor point of paragraph 1, wherein the second attachment portion includes both the aperture and the layer of adhesive.

7. The anchor point of paragraph 6, wherein the layer of adhesive extends entirely around the aperture.

8. The anchor point of paragraph 1, wherein the second attachment portion includes a flange.

9. The anchor point of paragraph 1, wherein the main body comprises a flexible piece of plastic.

10. The anchor point of paragraph 1, wherein the first attachment portion is formed integrally as a single piece with the second attachment portion.

11. A set of anchor points comprising:
the anchor point of paragraph 1, wherein the anchor point of paragraph 1 is a first anchor point; and
a second anchor point coupled to the first anchor point, wherein a perforation is disposed between the first anchor point and the second anchor point.

12. A surgical ablation probe comprising:
an umbilical;
a probe coupled to the umbilical; and
the anchor point of paragraph 1, coupled to the umbilical.

13. The surgical ablation probe of paragraph 12, wherein the anchor point is a first anchor point, wherein the surgical ablation probe includes a second anchor point coupled to the umbilical.

14. The surgical ablation probe of paragraph 13, wherein the first anchor point is coupled to the second anchor point, and wherein a perforation is disposed between the first anchor point and the second anchor point.

15. The surgical ablation probe of paragraph 12, further comprising a packaging, wherein the umbilical, the probe, and the anchor point are each disposed within the packaging.

16. The surgical ablation probe of paragraph 15, wherein the anchor point is a first anchor point, wherein the surgical ablation probe includes a second anchor point coupled to the umbilical and disposed within the packaging.

17. The surgical ablation probe of paragraph 16, wherein the first anchor point is coupled to the second anchor point, and wherein a perforation is disposed between the first anchor point and the second anchor point.

18. The surgical ablation probe of paragraph 15, wherein the packaging is a sterilized packaging.

19. The surgical ablation probe of paragraph 15, wherein the umbilical is a flexible coaxial cable.

20. The surgical ablation probe of paragraph 12, wherein the surgical ablation probe is a cryoablation surgical ablation probe.

21. An anchor point for an umbilical of a surgical ablation probe, the anchor point comprising:
a main body;
a first tab coupled to the main body and configured to flex relative to the main body about a first axis;
a second tab coupled to the main body and configured to flex relative to the main body about a second axis;
a third tab coupled to the first tab and configured to flex relative to the first tab about a third axis, wherein the first tab defines a third tab opening configured to be exposed when the third tab flexes relative to the first tab; and
a fourth tab coupled the second tab and configured to flex relative to the second tab about a fourth axis, wherein the second tab defines a fourth tab opening configured to be exposed when the fourth tab flexes relative to the second tab.

22. The anchor point of paragraph 21, wherein the first tab includes a first free end spaced opposite the first axis, the second tab includes a second free end spaced opposite the second axis, the third tab includes a third free end spaced opposite the third axis, and the fourth tab includes a fourth free end spaced opposite the fourth axis, wherein the first axis and the second axis are each positioned between the third free end and the fourth free end.

23. The anchor point of paragraph 21, wherein the first axis, the second axis, the third axis, and the fourth axis are all parallel to one another.

24. A surgical ablation probe comprising:
an umbilical;
a probe coupled to the umbilical; and
the anchor point of paragraph 21, coupled to the umbilical.

25. The surgical ablation probe of paragraph 24, wherein the first tab and the second tab are overlapped relative to one another, and are wrapped about a first portion of the umbilical.

26. The surgical ablation probe of paragraph 25, wherein the fourth tab is in an extended position through the third tab opening.

27. The anchor point of paragraph 21, further comprising a fifth tab coupled to the main body and configured to flex relative to the main body about a fifth axis, wherein the main body defines a fifth tab opening configured to be exposed when the fifth tab flexes relative to the main body, and a sixth tab coupled to the main body and configured to flex relative to the main body about a sixth axis, wherein the main body defines a sixth tab opening configured to exposed when the sixth tab flexes relative to the main body.

28. The anchor point of paragraph 27, wherein the first tab includes a first free end spaced opposite the first axis, wherein the fifth tab includes a fifth free end spaced opposite the fifth axis, and wherein the fifth free end faces the first free end.

29. The anchor point of paragraph 28, wherein the second tab includes a second free end spaced opposite the second axis, wherein the sixth tab includes a sixth free end spaced opposite the sixth axis, and wherein the sixth free end faces the second free end.

30. A surgical ablation probe comprising:
an umbilical;
a probe coupled to the umbilical; and
the anchor point of paragraph 27, coupled to the umbilical.

31. The surgical ablation probe of paragraph 30, wherein the first tab and the second tab are overlapped relative to one another, and are wrapped about a first portion of the umbilical.

32. The surgical ablation probe of paragraph 31, wherein the fourth tab is in an extended position through the third tab opening.

33. The surgical ablation probe of paragraph 32, wherein the sixth tab is in an extended position through the fifth tab opening.

34. The surgical ablation probe of paragraph 31, wherein the main body is overlapped, and is wrapped about a second portion of the umbilical.

35. The surgical ablation probe of clause 34, wherein the anchor point is formed from high density polyethylene.

Although various aspects and examples have been described in detail with reference to certain examples illustrated in the drawings, variations and modifications exist within the scope and spirit of one or more independent aspects described and illustrated.

What is claimed is:

1. An anchor point for an umbilical of a surgical ablation probe, the anchor point comprising:
a main body;
a first tab coupled to the main body and configured to flex relative to the main body about a first axis;
a second tab coupled to the main body and configured to flex relative to the main body about a second axis;
a third tab coupled to the first tab and configured to flex relative to the first tab about a third axis, wherein the first tab defines a third tab opening configured to be exposed when the third tab flexes relative to the first tab; and
a fourth tab coupled the second tab and configured to flex relative to the second tab about a fourth axis, wherein the second tab defines a fourth tab opening configured to be exposed when the fourth tab flexes relative to the second tab.

2. The anchor point of claim 1, wherein the first tab includes a first free end spaced opposite the first axis, the second tab includes a second free end spaced opposite the second axis, the third tab includes a third free end spaced opposite the third axis, and the fourth tab includes a fourth free end spaced opposite the fourth axis, wherein the first axis and the second axis are each positioned between the third free end and the fourth free end.

3. The anchor point of claim 1, wherein the first axis, the second axis, the third axis, and the fourth axis are all parallel to one another.

4. A surgical ablation probe comprising:
an umbilical;
a probe coupled to the umbilical; and
the anchor point of claim 1, coupled to the umbilical.

5. The surgical ablation probe of claim 4, wherein the first tab and the second tab are overlapped relative to one another, and are wrapped about a first portion of the umbilical.

6. The surgical ablation probe of claim 5, wherein the fourth tab is in an extended position through the third tab opening.

7. The anchor point of claim 1, further comprising a fifth tab coupled to the main body and configured to flex relative to the main body about a fifth axis, wherein the main body defines a fifth tab opening configured to be exposed when the fifth tab flexes relative to the main body, and a sixth tab coupled to the main body and configured to flex relative to the main body about a sixth axis, wherein the main body defines a sixth tab opening configured to be exposed when the sixth tab flexes relative to the main body.

8. The anchor point of claim 7, wherein the first tab includes a first free end spaced opposite the first axis, wherein the fifth tab includes a fifth free end spaced opposite the fifth axis, and wherein the fifth free end faces the first free end.

9. The anchor point of claim 8, wherein the second tab includes a second free end spaced opposite the second axis, wherein the sixth tab includes a sixth free end spaced opposite the sixth axis, and wherein the sixth free end faces the second free end.

10. A surgical ablation probe comprising:
an umbilical;
a probe coupled to the umbilical; and
the anchor point of claim 7, coupled to the umbilical.

11. The surgical ablation probe of claim 10, wherein the first tab and the second tab are overlapped relative to one another, and are wrapped about a first portion of the umbilical.

12. The surgical ablation probe of claim 11, wherein the fourth tab is in an extended position through the third tab opening.

13. The surgical ablation probe of claim 12, wherein the sixth tab is in an extended position through the fifth tab opening.

14. The surgical ablation probe of claim 11, wherein the main body is overlapped, and is wrapped about a second portion of the umbilical.

15. The surgical ablation probe of claim 14, wherein the anchor point is formed from high density polyethylene.

16. An anchor point for an umbilical of a surgical ablation probe, the anchor point comprising:
a main body having a first attachment portion and a second attachment portion coupled to the first attachment portion;
wherein the first attachment portion includes a loop configured to receive a portion of the umbilical;
wherein the second attachment portion includes at least one of an aperture that extends through the main body or a layer of adhesive on the main body.

17. The anchor point of claim 16, wherein the first attachment portion includes a lower hook and a slot, wherein the lower hook is configured to be inserted through the slot to form the loop.

18. The anchor point of claim 16, wherein the second attachment portion includes both the aperture and the layer of adhesive.

19. A set of anchor points comprising:
the anchor point of claim 16, wherein the anchor point of claim 16 is a first anchor point; and
a second anchor point coupled to the first anchor point, wherein a perforation is disposed between the first anchor point and the second anchor point.

20. A surgical ablation probe comprising:
an umbilical;
a probe coupled to the umbilical; and
the anchor point of claim 16, coupled to the umbilical.
